(19) United States
(12) Patent Application Publication
(10) Pub. No.: US 2010/0174052 A1
(43) Pub. Date: Jul. 8, 2010

(54) SEPARATION METHOD USING POLYMER MULTI PHASE SYSTEMS

(75) Inventors: Rolf Hjorth, Uppsala (SE); Karol Lacki, Uppsala (SE); Emmanuel Macedo, Uppsala (SE); Gunnar Malmquist, Uppsala (SE); Jamil Shanagar, Uppsala (SE); James Van Alstine, Uppsala (SE)

Correspondence Address: GE HEALTHCARE BIO-SCIENCES CORP.;PATENT DEPARTMENT, 101 CARNEGIE CENTER, PRINCETON, NJ, 08540, US

(73) Assignee: GE HEALTHCARE BIO-SCIENCES AB, UPPSALA (SE)

(21) Appl. No.: 12/663,089

(22) PCT Filed: Jun. 16, 2008
(86) PCT No.: PCT/SE08/00400

§ 371 Date: Dec. 4, 2009

(30) Foreign Application Priority Data
Jun. 19, 2007 (SE) 0701540-7

(51) Int. Cl. C07K 1/36

(52) U.S. Cl. 530/388.1; 530/387.1

(57) ABSTRACT

The present invention relates to a process of isolating one or more target compounds, wherein the clarification of feed is performed using partitioning in a multiphase system comprising a first polymer, which is a synthetic poly(acid), a second synthetic polymer, which is a poly(ether), and at least one salt, which clarification is followed by at least one step of affinity chromatography. The molecular weight of the poly(acid) may be in the range of 1000-100,000 Da. The target compound is preferably a biomolecule, such as a monoclonal antibody.

### CROSS-REFERENCE TO RELATED APPLICATIONS

This application is a filing under 35 U.S.C. §371 and claims priority to international patent application number PCT/SE2008/000400 filed Jun. 16, 2008, published on Dec. 24, 2008, as WO 2008/156409, which claims priority to patent application number 0701540-7 filed in Sweden on Jun. 19, 2007.

### FIELD OF THE INVENTION

The present invention relates to a process of isolating at least one target compound from a liquid, which process comprises at least one step of isolation performed by differentially partitioning said target between two aqueous phases which spontaneously form in the presence of certain polymers and added salts. The invention also encompasses a method of isolating a monoclonal antibody using such partitioning between two aqueous phases spontaneously formed in the presence of certain polymers and added salts; and kits for carrying out such partitioning.

### BACKGROUND OF THE INVENTION

The biotechnical revolution, including development of modern biopharmaceuticals and mapping of the human genome, has been made possible due to development of separation methods such as chromatography and electrophoresis. Such methods can be used in small scale as well as in large scale, and are known as flexible methods, being useful for a variety of substances including biological substances. However, they are demanding both technically and in terms of equipment. In addition, scaling of some processes such as electrophoresis results in a need for more complicated equipment due to nonlinear scaling of heating and cooling requirements.

Partitioning between the phases in aqueous polymer phase systems is an alternative method, which has been studied since the 1950's but whose commercial application has been severely restrained by lack of economically scalable phase systems. Together with separation methods such as crystallization and size exclusion; partitioning is considered a classic separation technique. It is related to differentially distributing a target and other substances between two phases. The term “partitioning” can refer to (a) liquid-solid partition such as in classic chromatography, (b) partitioning between two or more liquid phases (biphasic and multiphase system, respectively), (c) partitioning between a mobile liquid phase and another liquid phase immobilized at the surface of a solid phase support, and (d) partitioning of particles between a liquid phase and the phase interface between two phases. For the purposes of this patent application, partition and partitioning refer to situations such as b, c or d i.e. partitioning between liquid phases. Partition is typically expressed as a coefficient (K) related to the concentration in one phase versus another and for solutes K generally follows the Brønsted equation. Thus K is expected to vary exponentially with various types of interactions such as electrostatic and/or hydrophobic interactions, and also to be sensitive to solute size i.e. the area of interaction with liquid phases. In the case of interfacial partition, K is expected to vary exponentially with interfacial tension, which tends to localize particles to the phase interphase.

Classic two-phase systems are organic and aqueous two phase systems which normally have significant polarity differences between the phases, as well as significant interfacial tension. Such systems are not very useful for biologicals such as proteins or cells as they tend to be denatured by significantly apolar solutions and shear damage related to mixing of phase systems with significant interfacial tension. More useful for biologicals are the low tension, aqueous polymer two phase-systems. It is well recognized that the latter may contain some added organic solvents, e.g. ethanol, or other organic additives added to enhance target solubility, reduce liquid phase polarity, reduce foaming, act as bactericidal agents, etc.

Polymer two-phase systems can be formed by mixing certain hydrophilic and typically neutral polymers in aqueous solution. These include dextran (polyglucose) and poly(ethylene glycol) (PEG); as well as polysucrose (such as FICOLL™) and PEG; or linear polyacrylamide and PEG. Typical concentrations of each polymer are 5 to 10% w/w. At such concentrations, entropic forces tend to drive the formation of two phases both of which are typically greater than 90% (w/w) water but show subtle differences in polarity, hydrogen bond character, freezing point, etc. The phases are typically enriched in one polymer and have very low interfacial tension. In the biotechnical field, one advantage of the PEG and dextran type of two-phase system is that target proteins may partition in favour of the PEG-enriched phase while cell debris and some contaminants may partition to the interface or complementary phase.

WO 2004/020629 (Tjerneld) relates to the use of a PEG-like polymer comprising ethylene oxide (EO) groups in addition to propylene oxide (PO) groups, abbreviated as EOPO polymers. Such polymers, which show reverse thermal solubility, are known as “EOPO” polymers, and they are suggested in WO 2004/020629 for the separation of plasmids. At room temperature, the less dense, EOPO-enriched upper phase is isolated from the EOPO and dextran polymer aqueous two-phase system, and by a subsequent increase of its temperature to 37° C., the upper phase undergoes a further phase separation into a water-enriched phase and a self-associated EOPO polymer-enriched phase. Advantageously, the water-enriched phase should contain the desired plasmids. In general, this kind of EOPO and dextran systems offer advantages in terms of phase polymer component recycling and design of efficient two-stage partition separation process. However, a drawback is the cost involved in system formulation, which does not relate to the man-made synthetic polymer PEG but rather to the biologically derived and much more costly dextran.

Attempts to replace dextran with various starch or other polysaccharide polymers has resulted in limited success. One polymer, two-phase systems of intermediate interfacial tension can be formed by combining PEG and certain water structuring salts at relatively higher concentrations, e.g. 500 mM ammonium sulphate. PEG-salt two phase systems is one possible approach to overcome cost limitations but the increased PEG and salt concentrations create challenges which negatively impact process costs. These include viscous phases, salt reagent costs, salt disposal and equipment corrosion challenges, as well as target solubility issues which relate to capacity. As a consequence, the polymers are often difficult to recycle or otherwise have to be separated from the target via further downstream processing.

In the biotechnical field, polymer two phase systems, both in the forms with or without significant salt, are of general interest. This is because they are easily utilised in small as well as larger scale separations, without loss of efficiencies or dramatic changes in costs when scaling up to the larger volumes. Also, any standard separation approach, such as charge based, hydrophobicity based, affinity based, or size based separation, can be performed within a polymer two phase system. In general many undesired components, such as cell debris, endotoxins, nucleic acids will tend to appreciably partition to the lower (dextran-rich or salt rich, respectively) phase in a PEG and dextran or a PEG and salt two phase system. Thus, if a system can be found which provides for good target partition into the upper (PEG-rich) phase an effective primary separation and target concentration can be obtained.

Further, in efforts to overcome drawbacks related to interfacing in standard chromatographic and/or filtration processing, and to overcome the limitations of a single theoretical partition step per unit operation liquid-liquid partitioning two phase systems such as PEG-dextran or PEG-salt have been adapted to chromatographic uses by immobilising one phase on a chromatographic or other solid support capable of preferentially wetting that phase. The complementary phase is then pumped through the column offering repeated opportunities for equilibration between the mobile and stationary phase. This was commercially exploited by W. Müller et al. at Merck Darmstadt in the 1980's.

U.S. Pat. No. 5,093,254 (Giuliano et al) relates to an aqueous two-phase protein partitioning system is disclosed which employs polyvinylpyrrolidone as the upper phase and maltodextrin as the lower phase and provides a low-cost system for protein partitioning. The system can also be employed with the amion derivatives of chlorotriazine dyes, which bind in a noncovalent manner to the PVP and serve as a ligand for the proteins to be separated. It is stated that an advantage of this system is its cost-efficiency, as the dyes can easily be bound to the polymeric phase, without having to carry out the chromatographic and solvent extractions necessary to form the covalent bond in the PEG/hydroxypropyl starch system of the prior art. However, a drawback is the possible carcinogenic effect of such dyes.

Albertsson (P.-A. Albertsson, Partition of Cell Particles and Macromolecules, 2nd Edn., Wiley Interscience, N.Y., 1971. Chapter 10 Phase Diagrams, pp. 250-313) discloses systems comprising PEG and Na Carboxymethyl group modified dextran (CMD). The drawbacks of the system described is that (a) the polymer still involves an expensive polysaccharide; (b) the polymer is then further chemically modified; (c) the high molecular weight (Mw 2 200 000) and the inherent phase viscosity noted; and (c) the relatively high concentrations of polymers required to form phases, which is expected to bind water molecules and reduce system protein solubility.

Gupta et al (Vandana Gupta, Sunil Nath, Subhash Chand in Polymer 43 (2002) 3387-3390: Role of water structure on phase separation in polyelectrolyte-polyethyleneglycol based aqueous two-phase systems) relates to a study of the phase separation behaviour of polyelectrolyte-polyethyleneglycol (PEG) based aqueous two-phase systems (ATPS) carried out in order to elucidate the mechanism controlling phase-behaviour. It was concluded from this study that salt-assisted polymer-modified water structure interactions play a central role in phase separation in ATPS.

Saravanan (Settu Saravanan, Johny A. Reena, Jonnalagadda R. Rao, Thanapalan Murugesan, and Balanchandran U. Nair in J. Chem. Eng. Data 2006, 51, 1246-1249: Phase Equilibrum Compositions, Densities, and Viscosities of Aqueous Two-Phase Poly(ethylene glycol)+Poly(acrylic acid) Systems at Various Temperatures) relates to a study of the effect of temperature on the densities and viscosities of aqueous solution of poly(acrylic acid) (PAA) of different mass fraction (from 0.05-0.50) and liquid-liquid equilibrium, densities, and viscosities for the aqueous two-phase PEG-6000+PAA+water system at equilibrium.

As such there is still great need for novel separation methods, which are relatively technically simple and readily scaled.

### BRIEF DESCRIPTION OF THE INVENTION

One aspect of the present invention is to provide a method of separating biomolecules and other compounds, which provides high dynamic capacities and fast mass transfer. As defined in the appended claims, this may be achieved according to the present invention by partitioning said biomolecules and/or compounds into a volume, and not to an insoluble porous matrix whose surfaces provide for capture of target via controlled adsorption.

Thus, a specific aspect of the invention is to provide such a method that works also with colloidal particles, such as cells, chromosomes, etc., which are not amenable to chromatographic or filtration approaches where the solid support interferes or becomes clogged. This can be achieved by using specific polymer two phase systems according to the invention.

An additional aspect of the invention is to provide the use of such a polymer two phase system for the separation of biomolecules and other compounds, which system forms and phase separates spontaneously, and preferably also requires little complex equipment.

Another aspect of the invention is to provide a polymer two-phase system as such, which system has been optimised in terms of additives such as salt for efficient separation of biomolecules.

A further aspect of the invention is to provide a use of such a two-phase system as well as a kit comprising the optimised two-phase system according to the invention.

One or more aspects of the invention may be achieved as defined in the appended claims. Further aspects and advantages of the present invention will appear from the detailed disclosure that follows below.

### BRIEF DESCRIPTION OF THE DRAWINGS

FIG. 1 is a phase diagram for aqueous polymer two-phase systems of the present invention formed using PEG 4000 and NaPolyacrylate 8000, which is the sodium form of polyacrylic acid polymer. More specifically, the system was formed with 200 mM NaCl at 22° C. Binodal curve was estimated visually by titration of the system to concentration points related to circles: two-phase systems; squares: one-phase systems; and triangles: systems apparently at bimodal region and difficult to assign. The present phases form at relative low (total) polymer concentration and they are clear, of relatively low viscosity, and separate rapidly at unit gravity. Furthermore the phase binodal curve is more linear near the critical point, suggesting that two-phase system formed near this region will have significant tie-line length and therefore be more reproducible in terms of physical properties and also in terms of partition results. Note that the lowest total polymer concentration on the bimodal polymer concentration on the bimodal curve appears at approximately 12%, corresponding to 6% of each polymer.

FIG. 2 is a phase diagram of a two-phase system according to the invention comprising PEG 8000 and Na-polyacrylate 8000, which is the sodium form of polyacrylic acid polymer. The diagrams refer to systems formed with approximately 230 mM Na₂SO₄ (3% weight) at 25° C. Phase compositions determined; (circles) two-phase systems, (squares) one-phase systems, (triangles) systems apparently at bimodal region and difficult to assign. The present phases form at relative low (total) polymer concentration and are clear, of low viscosity, and separate rapidly at unit gravity. Furthermore the phase binodal curve is more linear near the critical point, suggesting that two-phase systems formed near this region will be more reproducible in terms of physical properties and also in terms of partition results. Note that the lowest total polymer concentration on the bimodal curve appears at approximately 10%, corresponding to 5% of each polymer, which in comparison with FIG. 1 is in keeping with the greater water structuring effect of the sodium sulphate salt.

FIG. 3 is a diagram showing the distribution in a two-phase system according to the invention of PEG 8000 rich phase (top) and NaPAA 15000 rich phase (bottom) at room temperature in systems containing 200 mM NaSulfate, and adjusted to pH 7. Systems are denoted as (x-y) where x is PEG wt % and y is NaPAA wt %. In this case the critical concentration lies somewhere between 4% and 4.5% of each polymer which allowing for the greater phase forming capability of the higher MW NaPAA polymer and the lower temperature, compares reasonably well with FIG. 2. A phase system of approximately equal phase volume was achieved. Similar results were seen for systems containing NaPAA 8000 (not shown).

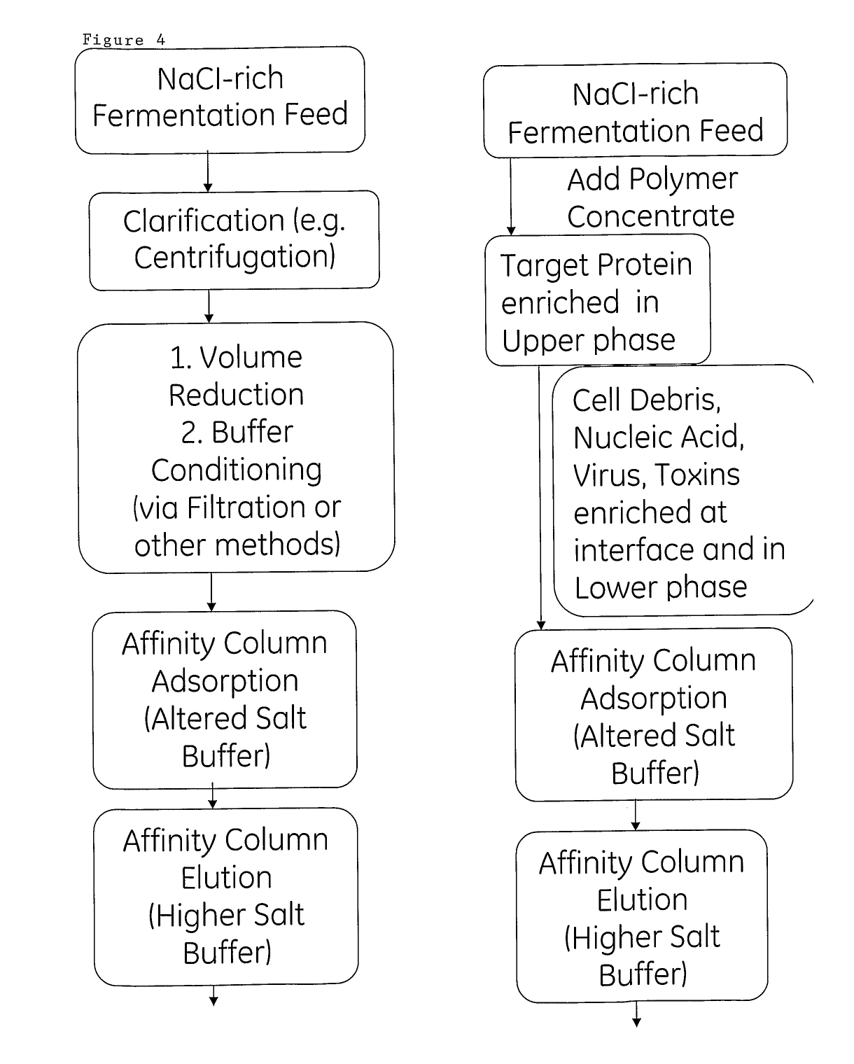

FIG. 4 is a comparative flow chart illustrating the differences between a process of the invention and a prior art process, as will be explained in Example 3 below.

FIG. 5 shows the partitioning of monoclonal antibodies and test proteins, as explained in Example 4 below.

### DEFINITIONS

The term “poly(acid)” as used in the present application means a linear or branched poly(acid) backbone containing a multitude of acidic groups as side groups and/or end groups.

The term “target compound” means herein compounds as well as molecules and cells, i.e. any entity which it is desired to isolate from a liquid.

### DETAILED DESCRIPTION OF THE INVENTION

The present invention relates to advantageous uses of an aqueous polymer two phase system for the isolation of target compounds, which are advantageously antibodies such as monoclonal antibodies, or Fab fragments of antibodies.

Thus, the invention relates a process of isolating one or more target compounds from an aqueous liquid, which comprises adding a liquid mixture capable of forming a multiphase system to a fermentation vessel, allowing a multiphase system to form, and isolating target compound(s) from one of the phases, which is rich in one of the added polymers.

In one embodiment, the liquid mixture added comprises a first polymer, which is a synthetic poly(acid), a second synthetic polymer, which is a hydrophilic poly(ether), and at least one salt. Such polymers will be discussed in more detail below.

In another embodiment, the present process, including the fermentation and the partitioning between phases, is carried out in a plastic bag, optionally connected to a moving platform, such as a rocking platform. Suitable plastic bags are readily available on the market, e.g. from Wave Biotech. In an alternative embodiment, the fermentation is carried out in a different container and the fermentation is then directly transferred to a plastic bag for partitioning in the multiphase system, without any intervening purification steps.

In an advantageous embodiment, the target compound is an antibody, such as a monoclonal antibody or an antibody fragment, such as a Fab fragment. In an alternative embodiment, the target compound is a fusion protein comprising an antibody or a fragment thereof.

In one aspect, the present invention is a process of isolating one or more target compounds, wherein the clarification of feed is performed using partitioning in a multiphase system comprising a first polymer, which is a synthetic poly(acid), a second synthetic polymer, which is a hydrophilic poly(ether), and at least one salt, which clarification is followed by at least one step of affinity chromatography.

The feed may be any liquid wherein a target compound has been produced, such as a fermentation broth or a biological fluid. If required, the process includes a step of lysing cells producing target compound before the clarification in a two phase system according to the invention.

In one embodiment, the affinity chromatography comprises binding to Protein A ligands. Protein A chromatography is a well known method, and is in this context understood to encompass adsorption to any resin which comprises recombinant or native Protein A; parts of Protein A or any other modified form of Protein A which has retained its selectivity towards antibodies. Commercially available Protein A resins comprises e.g. the MABSELECT™ family (GE Healthcare).

The specific details of the partitioning of target compound between the phases of a multiphase system will be discussed in detail below.

The affinity step may be followed by one or more additional chromatography steps and optionally steps for virus removal. In one embodiment, the affinity chromatography is followed by ion exchange and/or hydrophobic interaction chromatography (HIC). Anion exchangers, cation exchangers and HIC resins are well known and commercially available. In an advantageous embodiment, at least one subsequent step is an ion exchange, taking advantage of the fact that the poly(acid) is charged.

In another embodiment, the affinity chromatography is followed by multimodal ion exchange chromatography. Multimodal ion exchange is also well known, and utilises a ligand that comprises more than one functional group such as an ion exchanging group in close proximity to a hydrophobic group. Illustrative examples are CAPTO™ MMC and CAPTO™ Adhere (GE Healthcare).

The target compound isolated in the present process may e.g. be a biomolecule such as a protein, peptide, nucleic acid, cell, virus, or any part, fragment or fusion product of anyone of the above. Thus, in one embodiment, the target compound is an antibody, or a fragment or fusion product thereof. Illustrative antibody fragments are e.g. Fab fragments. In another embodiment, the target compound is a nucleic acid, such as DNA or RNA, e.g. a plasmid, genomic DNA, an aptamer or an oligonucleotide. In an additional embodiment, the target compound is a cell, such as a eukaryotic or a prokaryotic cell, for example an adult cell or a progenitor cell. Thus, in one embodiment of the present process, the target compound is a biomolecule, such as an antibody, preferably a monoclonal antibody. In another embodiment, the target compound is a Fab fragment.

In a second aspect, the present invention is a method of isolating at least one antibody from a liquid, which method comprises partitioning in a multiphase system comprising a first polymer, which is a synthetic poly(acid), a second synthetic polymer, which is a hydrophilic poly(ether), and at least one salt. In an advantageous embodiment, the molecular weight of the poly(acid) is in the range of 1000-100,000 Da.

The multiphase system used in this aspect of the invention may be as discussed above in the context of the process and the multistep process of the invention, and will as mentioned be discussed in greater detail below.

In an advantageous embodiment of the present method, the antibody is a monoclonal antibody, which is recovered from the upper phase of the system. Thus, in a specific embodiment, the multiphase system used to isolate the antibody such as a monoclonal antibody is an aqueous polymer two phase system comprising about 4-8% polyethylene (PEG), such as 6% PEG, and 4-8% poly(acid), such as about 6% poly(acid), with 20 mM salt present. The PEG may be PEG 8000, and the poly(acid) may be NaPAA 1500.

The skilled person can easily optimise the pH for the present isolation. In an advantageous embodiment for the isolation of an antibody, the pH is about neutral.

The present invention provides an advantageous method for separating an antibody such as a monoclonal antibody from a feed comprising several contaminants such as DNA and RNA. In one embodiment, the antibody is purified from DNA and RNA which both partition to the lower phase.

The step of partitioning target compound, which partitioning is used in both the first and the second aspect of the invention, will now be described in detail.

The polymers used in the liquid mixture and multiphase system used in the present invention are aqueous in the sense that they form aqueous phases when combined with water. Further, as understood by the skilled person, in the present context the term liquid “mixture” refers merely to a combination of the herein-defined components. Under which conditions such liquid mixtures exist as one, two or more phases is deducible from phase diagrams. One advantage of the liquid mixtures of the invention is that they give rise to phases which appear less viscous, optically clearer and faster separating than many commonly studied phase systems.

In one embodiment, the molecular weight the poly(acid) polymer is in the range of 900-100,000 Da, such as 1000-20,000 Da. In one embodiment, the molecular weight is in the broad range of 400-1,000,000 Da. In one embodiment, the poly(acid) used to isolate the monoclonal antibody is selected from the group consisting of poly(acrylic acid) and poly(methacrylic acid).

The poly(acid) may be any suitable synthetic poly(acid). Thus, the backbone may be a hydrocarbon chain, a poly(ether), a polyester, a polyamide, a polyacetal, a polyurethane or a polysulfone. In one embodiment, the poly(acid) comprises a hydrocarbon (vinyl polymer) or a poly(ether) chain, to which acidic groups have been coupled. The skilled person can easily prepare such poly(acid)s.

Thus, in one embodiment of the present liquid mixture, the poly(acid) is selected from the group consisting of polymers formed using acid-functional monomers such as acrylic acid, methacrylic acid, itaconic acid, crotonic acid, maleic acid, fumaric acid, vinylbenzoic acid, acrylamidoglycolic acid, acryloyloxyethyl succinate, vinylsulfonic acid, styrenesulphonic acid, acrylamidomethylpropanesulfonic acid, vinylphosphonic acid etc. In an advantageous embodiment, the poly(acid) is poly(acrylic acid) (PAA) or polyacrylate. When used in a polymer multiphase system, the PAA rich phase will be clear, separate rapidly and exhibit lower viscosity than dextran-based systems. Liquid mixtures capable of forming PAA-based multiphase systems according to the invention are readily formed by combining e.g. 40% solutions of commercially available NaPAA with an hydrophilic poly(ether) and salt. The poly(acid) may be in acidic, anhydride or deprotonated form, i.e. the salt form.

In one embodiment, the molecular weight the hydrophilic poly(ether) is in the range of 900-100,000 Da, such as 1000-20,000 Da. In one embodiment, the molecular weight is in the broad range of 400-1,000,000 Da.

In an advantageous embodiment, the poly(ether) is selected from the group consisting of water soluble poly(ether)s which includes poly(ethylene)glycol (PEG); ethylene oxide propylene oxide (EOPO) in either random copolymer form (e.g. BREOX® polymers) or block copolymers (e.g. PLURONIC® polymers) and may include variously modified forms of such polymers (e.g. monomethoxy forms of PEGs). In an advantageous embodiment, the ethylene oxide polymer is PEG. In the separation of biomolecules, PEG is often favoured by target localization as it is biocompatible and an accepted FDA excipient; and because it can be readily separated from proteins, cells and other targets. In an alternative embodiment, the poly(ether) is EOPO. As is known by the skilled person, EOPO separates into two phases upon heating and is consequently regarded a thermoseparating polymer. Thus, this embodiment, the present system may form a three phase system.

As the skilled person will understand, the present synthetic poly(acid) and poly(ether) are chosen to be able to form an aqueous two phase system in the presence of salt. The skilled person can easily deduce, based on phase diagram, at which pH values, salt concentrations, molecular weights etc the said polymers will exist in the system as one phase or as more. Thus, in one embodiment of the present system, the poly(ether) is capable of forming a system of two physically different phases in the presence of the poly(acid) and salt, wherein each phase is enriched in one of the polymers.

Thus, the skilled person can easily decide suitable conditions such as pH and temperature at which a multiphase system, such as a two phase system, is formed from the present liquid mixture based on phase diagram data and optionally very simple routine experimentation. In one embodiment, the pH value of the present liquid mixture is close to neutral. The temperature may be in the range of 4-30° C., such as room temperature, for forming a two phase system. If a third phase is to be formed from a thermoseparating polymer rich phase, then higher temperatures are used at that stage.

The two-phase systems used in the invention which comprise poly(ether) and acid-group containing polymers may contain other charged and noncharged groups, as is the case with two phase systems formed with PEG and poly(vinylmethylether-co-maleic anhydride) by the present inventors.

In a specific embodiment of the present system, the salt concentration is in the range of 1-500 mM, such as below 300 mM or in the range of 100-300 mM. As the skilled person will understand the amount of salt needed to form a two-phase system will be influenced by polymer MW, concentration and physical status. Thus only 100 mM buffer salt is required to form a two-phase system if it is formulated with the sodium or other salt form of the poly(acid).

In an advantageous embodiment, the salt is selected from the group consisting of NaCl, Na₂PO₄, KPO₄, NaSO₄, potassium citrate, (NH₄)₂SO₄, sodium acetate and combinations thereof. The skilled person can easily predict the effects of each specific salt on phase separation, such as the isolation of a protein, based on the Hoffmeister series. This because it is well known that salts at the lower end of the Hoffmeister series, such as NaCl, will tend to shift a net positively charged protein towards the ethylene oxide polymer rich phase; while a salt which is present more to the upper or right end of the Hoffmeister series instead will shift said protein towards the poly(acid) rich phase. In one embodiment, the present liquid mixture comprises 10% or less of salt(s).

The total polymer concentration of the present liquid mixture can be optimised for each envisaged use. For example, it is well known that proteins and other macromolecules can be precipitated out of solution by addition of relatively high amounts of water soluble polymers. Therefore, if the system according to the invention is to be used in protein separation, too high a total polymer concentration would not allow for sufficient protein solubility to achieve a cost efficient separation. Thus, in one embodiment of the present liquid mixture, which is advantageous for the isolation of biomolecules and/or particles, the total polymer content constitutes about 8-20% (w/w) of the system. In one embodiment, the liquid mixture comprises 10-20% (w/w). In another embodiment, the liquid mixture comprises about 70% of water.

Thus, in one embodiment, the present liquid mixture comprises about 4-6% of each polymer, such as about 5%, about 4.5% or about 4% of each polymer. In another embodiment, the liquid mixture comprises up to about 10% of each polymer, such as about 8% of each polymer.

In a specific embodiment, the multiphase system comprises one or more chromatography ligands. Such chromatography ligands may be used as a tool when applying the present liquid mixture to isolation of biomolecules or particles, in which case the ligands may bind a certain target compound partition said target compound to the phase favoured by the ligands. In one embodiment, the ligands are affinity ligands, which are capable of binding target molecules by highly specific interactions of the “lock/key” type, such as between receptor and ligand, or antibody-antigen. Illustrative affinity ligands are e.g. Protein A or Protein A-based ligands. In an advantageous embodiment, the affinity ligands are polymer-modified to facilitate their partitioning to a specific phase. In another embodiment, polymer-modified affinity ligands are added to partition interacting targets into the phase enriched in a polymer most similar to that linked to the ligand.

A third aspect of the present invention is a kit for the isolation of at least one antibody, such as a monoclonal antibody, which kit contains a liquid mixture or multiphase system as described above. In one embodiment of the kit, the liquid mixture or multiphase system is provided in a plastic bag.

In an advantageous embodiment, the present kit comprises at least polymer, which is a synthetic poly(acid), in aqueous solution or in dry form.

### EXAMPLES

The present examples are provided for illustrative purposes only, and should not be construed in any way as limiting the invention as defined by the appended claims.

### Example 1

### Preparation of Two Phase System According to the Invention and Phase Diagram

### Materials

Polymers: Poly(ethylene glycols) 4000 (Merck), PEG 8000 (Sigma-Aldrich), Na-poly(acrylates) from Aldrich, CAS-number: 9003-04-7, Molecular weight 30000 (in 40 wt % water solution), Molecular weight 8000 (in 45 wt % water solution). NaCl and Na₂SO₄ Methanol, Ba(NO₃)₂ (from Merck and P.A. quality). Millipore water was used in all solutions.

### Determination of Phase Diagram

The phase boundary of the stem (bimodal) was determined by the titration method well known methods [see Methods in Enzymology, Vol. 228, Aqueous Two-Phase Systems, Harry Walter and G. Johansson eds. Academic Press, New York, 1994]. In this case a system having a composition that is suspected to lie within the two-phase region is made. If the system turns turbid on mixing it indicates the existence of a two-phase system. Upon addition of a salt solution having the same salt concentration as the studied system the system becomes diluted with respect to the polymers. If the polymer concentration falls below a critical value the system turns into a one phase system, which does not become turbid on mixing. By adding polymers and diluting the system sequentially the phase diagram is mapped on both sides of the phase boundary, i.e. the binodal curve. The systems were determined in 22° C. (room temperature) and 25° C. (water bath).

### Refractive Index Measurements

The refractive index of a solution is a linear additive property in water rich solutions (>90%). By making separate standard curves of known concentrations of PEG-water, Na-poly(acrylate)-water, and salt-water solutions. The refractive index instrument was obtained from Carl Zeiss (Oberkochen, Wurttemberg, Germany).

### Determination of PEG

Since the solubility of salt and Na-poly(acrylate) is very low in methanol (<0.1 wt %) while PEG have a very high solubility, it is possible to selectively extract PEG into methanol and determine the PEG gravimetrically by evaporating the methanol. 1.00 g of a top or bottom phase is mixed with at least 6 g Methanol. A precipitate of Na-polyacrylate and salt is formed and is centrifuged at 3000×g for 10 min. The supernatant containing PEG is collected and placed in 15 ml glass tubes whose height are known. A further wash of the precipitate with 2 g Methanol with subsequent centrifugation is performed. This later supernatant fraction is pooled with the first one. The tubes are allowed to stay open in a vented hood for 3 days. Most of the Methanol is evaporated and the remaining is evaporated in an oven at 70° C. The tubes are weighed and the dried PEG is determined gravimetrically.

### Determination of Na₂SO₄

The sodium sulfate salt can be determined by titration with Ba-sulfate. However, since the Na-polyacrylate is precipitated by divalent cations, this polymer must be removed before analysis. This is done by the following:

1 g sample (top- or bottom phase was added to a 15 ml glass tube (A). 0.1 g Na-polyacrylate 8000, (concentration: 45 wt % in water) and 0.3 g PEG 8000 (concentration 30 wt % in water) and 0.2 g HCL (37 wt %) was added to the sample and vortexed and finally centrifuged at 3000×g. A two-phase system was formed containing a bottom viscous phase composed by PEG and polyacrylic acid in high concentrations. The top-phase is a water rich phase. The volume ratio is high (>10). The water rich phase is collected carefully and placed in another 15 ml glass tube (B) with known weight. 1.5 g is added to the viscous phase in the glass tube (A) vortexed and centrifuged at 3000×g. The water rich phase is again carefully collected and placed in tube (B). In this procedure the PEG and polyacrylic acid is removed separated from the water solution containing all the Na₂SO₄.

1.5 g of warm 13 wt % Ba(NO₃)₂ water solution is now added to tube (B) containing sulfate salt and a precipitate of BaSO₄ is immediately formed. The tube (B) is centrifuged at 3000×g and the supernatant is discarded. This is repeated 3 times to remove traces of soluble material. The tube (B) with the BaSO₄ precipitate is dried in an oven at 70° C. 3 days. The amount of BaSO₄ is determined gravimetrically and the concentration of Na₂SO₄ can then be calculated.

### Determination of Na-poly(acrylate)

The concentration of Na-polyacrylate is determined through refractive index (R1) by the following method. The R1 of a phase that is diluted 3 times is determined. This value contains contribution from PEG and salt. From known concentration of PEG and Salt determined as described their contribution to the refractive index is determined. The resultant value is due to the Na-polyacrylate that is determined by the previously determined standard curves.

### Phase Separation

The aqueous polymer phase systems are prepared according to standard for such systems. In order to get around the time needed for dry polymers to become fully hydrated, which in undisturbed solutions can take 24 hours, stock solutions of typically 30 to 40 weight % of the polymers are compounded. In the case of NaPAA it is possible to commercially purchase such stock solutions. In the case of PEG the stock solutions were compounded by the operator. So too stock solutions of NaCl (1 M) or other salts, e.g. 0.5M NaPhosphate pH 6.8, are also compounded. To make a 1000 gram (approximately 1 litre) phase system consisting of 6% PEG, 6% NaPAA, 300 mM NaCl, 50 mM NaPhosphate one simply mixes 150 g of PEG stock, 150 g of NaPAA stock, 300 g of NaCl stock and 100 g of NaPhosphate stock then adds water to the desired total. Once compounded such a system can be stirred for a few minutes to ensure full mixing and then allowed to spontaneously separate into two phases. To make 10 ml of the same system would simply require 100 times smaller amounts of each stock.

The two-phase systems are formed by mixing stock solutions of PEG, Na-poly(acrylate) and salt into 12 ml graded glass tubes. The total weight of the systems is 10 g. The systems are mixed up-side-down ca 15 times and the system becomes completely turbid. The system is then allowed to separate in a water bath at 25° C. The systems are generally completely separated within 30 min. However, the systems were allowed to stand for 1-2 hours. The refractive index of the phases is very similar so that it may be difficult to discover the interface. The separated phases are clear and have relatively low viscosity (visually observed).

Table 1 below provides information related to various tested PEG 4000 or 8000 and NaPAA 8000 or 30000 and NaCl or NaSulfate containing two phase systems. The PEG4000 and NaPAA 8000 two phase system at 22° C. is close to 5.28 and 5.68% respectively which is in keeping with the lower PEG polymer MW. By comparison, the PEG 4000 and NaPAA 30000 critical concentration is approximately 4.7 weight % of each polymer.

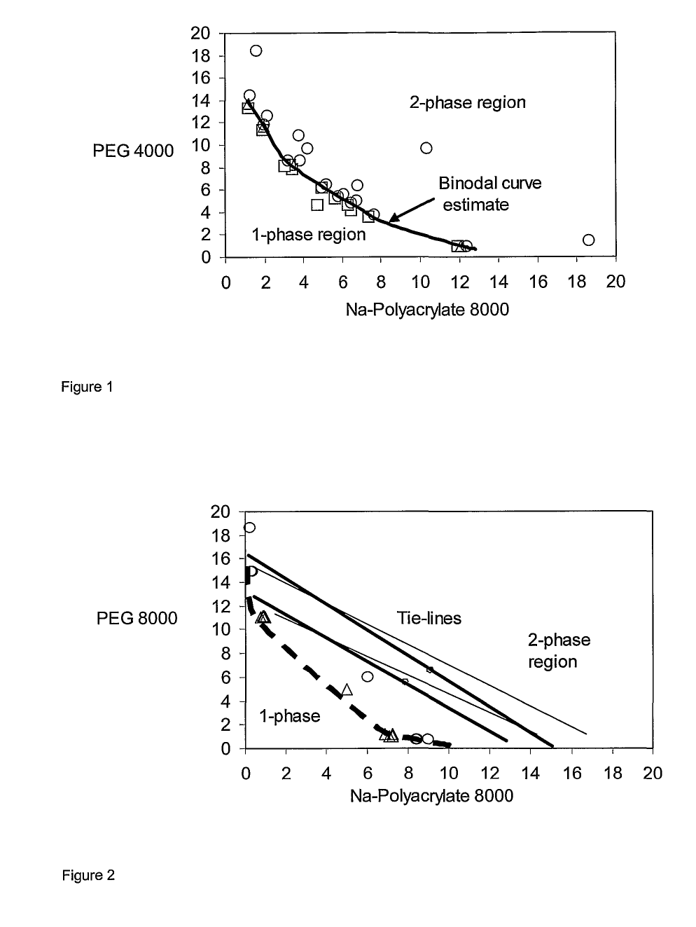

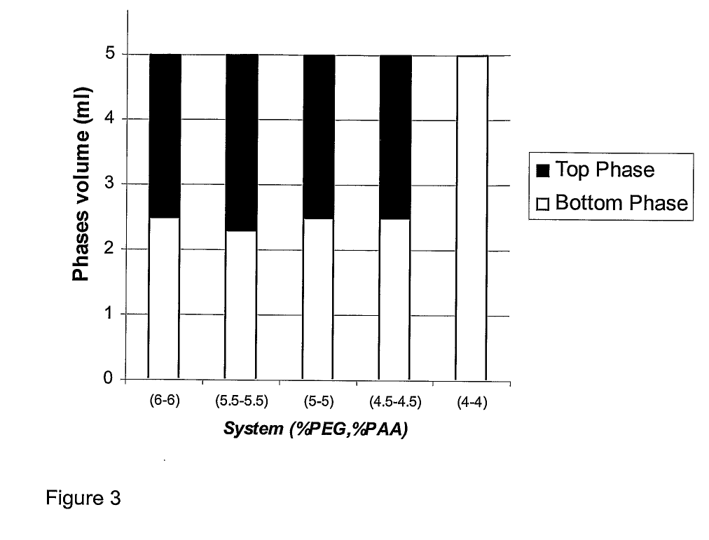

TABLE 1
PEG 4000 or 8000 and NaPAA 8000
or 30000 Two-Phase Systems*

| | | | Phase Separation noted |
|---|---|---|---|
| | Na- | Salt type and | after 2 hours at |
| PEG | poly(acrylate) | concentration | given temperature |
| Mw 4000, | Mw 30000, | NaCl | ~22° C. Apparently at |
| 4.7 wt % | 4.7 wt % | 150 mM | bimodal and close to |
| | | | critical concentration** |
| Mw 4000, | Mw 8000, | NaCl | ~22° C. Two-phase |
| 5.42 wt % | 5.77 wt % | 200 mM | system close the binodal |
| | | | (see FIG. 1) |
| Mw 4000, | Mw 8000, | NaCl | ~22° C. One-phase |
| 5.28 wt % | 5.68 wt % | 200 mM | system close to binodal |
| Mw 8000, | Mw 8000, | Na₂SO₄ | 25° C. Two-phase |
| 5.00 wt % | 5.00 wt % | 230 mM | system (see FIG. 2) |

*pH ~7.5, 1.05 wt % NaCl is approx. 150 mM, 3.00 wt % Na₂SO₄ is approximately 230 mM.
**Polymer concentration below this or salt concentration below this leads to system that does not appear to separate into two phases over two hour period.

### Example 2

### Effect of Salt and pH on Two Phase System According to the Invention

Two phase systems were prepared as described above. The effect of pH on EOPO 3900 and NaPAA 15000 systems was studied. The results are shown in Table 2, which provides insight into the effect of pH on phase volume ratios and phase system formation in EOPO and NaPAA containing two-phase systems in 200 mM NaP buffer. At these polymer molecular weights, concentrations and salt conditions, two phases were formed at pH 6 to 8 but not at pH 5.

TABLE 2
Effect of salt and pH on EOPO 3900 NaPAA15000 two phase systems

| | | | | | Upper | Bottom |
|---|---|---|---|---|---|---|
| EOPO | PAA | mM NaP, | Phase | Volume | EOPO- | NaPAA- |
| 3900 | 15000 | pH 7 | Forms | Ratio | rich phase | rich phase |
| 4 | 4 | 0 mM | − | | | |
| | | pH 7 | | | | |
| 4 | 4 | 100, pH 7 | − | | | |
| 4 | 4 | 200, pH 7 | + | 0.35 | clear | clear |
| 4 | 4 | 300, pH 7 | + | 0.28 | clear | Turbid |
| 4 | 4 | 400, pH 7 | + | 0.22 | clear | Turbid |
| 4 | 4 | 200 pH 5 | − | | | |
| 4 | 4 | 200 pH 6 | + | 0.35 | clear | clear |
| 4 | 4 | 200 pH 8 | + | 0.35 | clear | clear |

### Example 3

### Protein Purification Process

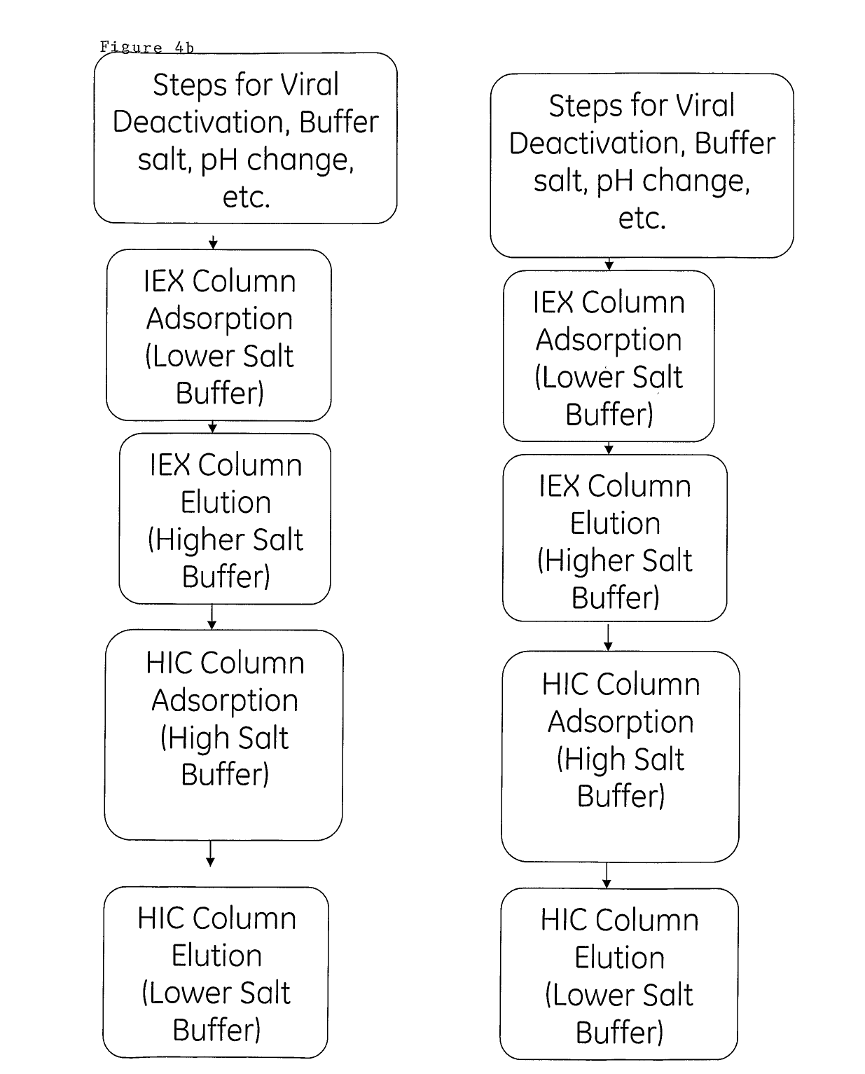

In this example, it is explained how a protein may be purified using the process outlined in FIG. 4. More specifically, a two-phase polymer system is used for clarification of sample in a step preceding the affinity chromatography.

### Example 4

### Partitioning of Monoclonal Antibody (MAb) in Polymer Two Phase System

Two different monoclonal antibodies (MAb1 and MAb2) as well as polyclonal IgG were partitioned in this example. Table 1 below shows how it is possible to form a wide variety of NaPAA based poly(acid) two phase systems, which show interesting partitioning of a wide variety of antibodies (Abs) and monoclonal antibodies (Mabs). It should be noted that protein solubility in these systems can be up to 5 g/L.

TABLE 3
Partition of Mabs in EOPO3900- or PEG8000-NaPAA 15000 systems
containing 6% (w/w) of each polymer

| | | | | | Phase | | | K |
|---|---|---|---|---|---|---|---|---|
| | EOPO | PEG | | NaCl | Volume | K | K | Polyclonal |
| # | MW | MW | pH | (mM) | Ratio | Mab 1 | Mab 3 | IgG |
| 1 | 3900 | | 7 | 200 | 0.85 | 0.2 | 0.2 | |
| 2 | | | 7 | 300 | 0.9 | 2.3 | 1.2 | |
| 3 | | 8000 | 7 | 200 | 1.4 | 0.2 | 0.7 | |
| 4 | | | 7 | 300 | 1.5 | 2.1 | 2.3 | |
| 5 | | | 7 | 400 | 1.5 | Some precipitation | | |
| 6 | | | 8 | 600 | 1.8 | Some precipitation | | |
| 7 | | 4000 | 6 | 400 | 1.5 | | | |
| 8 | | | 8 | 400 | 1.6 | | >99 | |
| 9 | | | 8 | 600 | 1.8 | | >99 | |
| 10 | | 1500 | 6 | 400 | 2.2 | | 28 | |
| 11 | | | 8 | 400 | 2.1 | | | >99 |
| 12 | | | 8 | 600 | 2.4 | | 61 | |
| 13 | | 900 | 8 | 400 | 4.0 | | 71 | |

Notes
A. K = [Protein in upper phase]/[Protein in lower phase].
B. Systems buffered with 10 mM NaP pH 7 or 20 mM NaP at pH 6 or pH 8.
C. All systems at RT except 13 which formed two-phases at 4° C.
D. EOPO-PAA systems formed water-, EOPO- and PAA-rich 3 phase system at 37° C.
E. Differences in K values greater than 99 (99% protein in upper phase) not significant.
F. Mab 1 pI > 8, Mab 3 pI 6 to 8 switch relatively more hydrophobic character than Mab 1. Similar partition results seen for polyclonal human IgG sample (Gammanorm, Octapharma).
G. All systems but 11 studied with 0.2 mg IgG per ml system (approx. 0.4 mg/ml upper if K = 70).
H. Slight, fine, precipitation noted with IgG in systems 4, 7, 8, and 9. Assume buffer adjust would eliminate.
I. System 11 with 2.5 mg IgG per ml of system (~6 mg/ml upper phase at 76% recovery) showed some fine precipitation. Doubling the IgG load reduced recovery in upper phase to 66%. Salt and pH adjustment might increase solubility for Ig's.
J. Control study based on above Mabs and system from Andrews et al. (Bioseparation 6, 303-313, 1996) (PEG1450/KPhosohate/NaCl: 15/14/12%) gave Mab 2 K of 90 and solubility limit of <1 mg/ml system in keeping with published results.

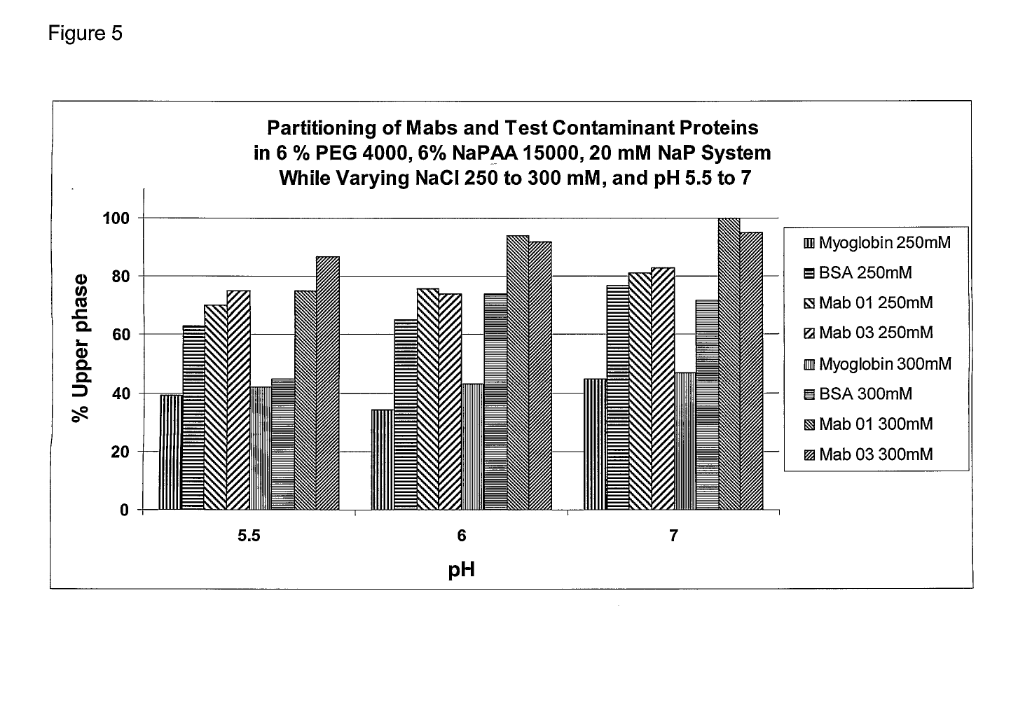

Also, FIG. 5 shows how partition of Mabs and two test contaminant proteins is altered according to the invention with pH and salt concentration in PEG4000 NaPAA 15000 two phase systems. The two test proteins—whale myoglobin and bovine serum albumin (BSA) are commonly used in studies to mimic how host cell protein contaminants might partition in processes for large scale purification of Mabs. It can be seen that conditions can be found where the myoglobin and BSA proteins show reduced upper phase partition (e.g. 40 to 60%). Thus a first partition step might be expected to not only partition and concentrate a Mab or other protein target into the PEG-rich upper phase but also significantly reduce protein contaminants.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A process of isolating one or more target compounds from an aqueous liquid, which comprises combining a liquid comprising a first polymer which is a poly(acid), a second polymer which is a poly(ether), and at least one salt with a fermentation broth in a container, allowing formation of at least two phases, and isolating target compound(s) from one of the phases, which is rich in one of the polymers.

2. The process of claim 1, wherein the fermentation broth comprises cells expressing at least one target, or cell debris from such cells, and wherein the container is the container wherein the fermentation was carried out.

3. The process of claim 1, wherein the target compound is an antibody, such as a monoclonal antibody or an antibody fragment, such as a Fab fragment.

4. The process of claim 1, wherein at least one of the first and the second polymer is a synthetic polymer.

5. A multistep process of isolating one or more target compounds, wherein the clarification of feed is performed using partitioning between the phases of a multiphase system comprising a first polymer, which is a poly(acid), a second polymer, which is a hydrophilic poly(ether), and at least one salt, which clarification is followed by at least one step of affinity chromatography.

6. The process of claim 5, wherein the molecular weight of the poly(acid) is in the range of 1000-100,000 Da.

7. The process of claim 5, wherein at least one of the first and the second polymer is a synthetic polymer.

8. The process of claim 5, wherein the partitioning in the multiphase system is carried out in a plastic bag, optionally connected to a moving platform, such as a rocking platform.

9. The process of claim 5, wherein the affinity chromatography comprises binding to Protein A ligands.

10. The process of claim 5, wherein the affinity chromatography is followed by one or more steps comprising ion exchange and/or hydrophobic interaction chromatography and/or multimodal ion exchange chromatography.

11. The process of claim 9, wherein the target compound is an antibody, such as a monoclonal antibody or an antibody fragment, such as a Fab fragment.

12. A method of isolating at least one antibody from a liquid, which method comprises a step of partitioning between the phases a multiphase system comprising a first polymer, which is a synthetic poly(acid), a second synthetic polymer, which is a hydrophilic poly(ether), and at least one salt.

13. The method of claim 12, wherein the molecular weight of the poly(acid) is in the range of 1000-100,000 Da.

14. The method of claim 12, wherein the antibody is a monoclonal antibody or a Fab fragment, which is recovered from the upper phase.

15. The method of claim 12, wherein the multiphase system is an aqueous polymer two phase system comprising about 4-8% polyethylene (PEG), such as 6% PEG, and 4-8% poly(acid), such as about 6% poly(acid), with 20 mM salt present.

16. The method of claim 15, wherein the PEG is PEG 8000.

17. The method of claim 15, wherein the poly(acid) is NaPAA 1500.

18. The method of claim 12, wherein the pH is about neutral.

19. The method of claim 12, wherein the antibody is purified from DNA and RNA which both partition to the lower phase.

20. The method of claim 12, wherein the poly(acid) is selected from the group consisting of poly(acrylic acid) and poly(methacrylic acid).

21. The method of claim 12, wherein the molecular weight of said other poly(ether) is in the range of 900-100,000 Da.

22. The method of claim 12, wherein the poly(ether) is selected from the group consisting of poly(ethylene)glycol (PEG); ethylene oxide propylene oxide (EOPO); Breox™; Pluronic™; and ethoxy-containing polysaccharides.
